(19) United States
(12) Patent Application Publication
(10) Pub. No.: US 2008/0216214 A1
(43) Pub. Date: Sep. 11, 2008

(54) Multi-function headband

(76) Inventor: Timothy S. Dolby, Grand Junction, CO (US)

Correspondence Address: RICHARD L. MILLER, 12 PARKSIDE DRIVE, DIX HILLS, NY, 11746, US

(21) Appl. No.: 11/714,024

(22) Filed: Mar. 5, 2007

(51) Int. Cl. A42B 1/06; A42C 5/02; A61F 9/02

(52) U.S. Cl. 2/410; 2/181; 2/209; 2/452

(57) ABSTRACT

A multi-function headband, including a head band, a pair of ear muffs, a neck protector, and apparatus for replaceably maintaining the head band around the head of a user. The head band encircles the head of the user. The pair of ear muffs depend from the head band and overlie and protect the ears of the user, respectively. The neck protector depends from the head band and overlies and protects the back-of-the-neck of the user.

### 1. BACKGROUND OF THE INVENTION

A. Field of the Invention

The embodiments of the present invention relate to a headband, and more particularly, the embodiments of the present invention relate to a multi-function headband.

B. Description of the Prior Art

Numerous innovations for headbands have been provided in the prior art that will be described below. Even though these innovations may be suitable for the specific individual purposes to which they address, however, they differ from the embodiments of the present invention in that they do not teach a multi-function headband.

(1) U.S. Pat. No. 2,560,267 to Baker et al.

U.S. Pat. No. 2,560,267 issued to Baker et al. on Jul. 10, 1951 in class 132 and subclass 46 teaches a hair band including an elongated strip of flexible fabric of double ply of sufficient length to encircle the brow and lower rear portion of the scalp, apparatus to secure together the opposite ends of the strip to form a band adapted to encircle the head, and an elongated flexible resilient clamping element positioned between the plies and extending lengthwise of the strip across the brow portion and having its ends located about midway of the length of the band when the ends of the strip are connected.

(2) U.S. Pat. No. 4,486,903 to Krystal.

U.S. Pat. No. 4,486,903 issued to Krystal on Dec. 11, 1984 in class 2 and subclass 197 teaches an adjustable cap with a back opening and a strip of adjustable length whereby the cap can be adjusted as to size having an inband that can be stored in the head cover or folded downwardly to cover the ears. The inband pattern has an upper edge that curves downwardly on each side of a center portion so that when it is sewn into the cap, the end portions of the inband are stressed to bias them inwardly to hug the head and ears in use.

(3) U.S. Pat. No. 4,833,734 to Der Estephanian.

U.S. Pat. No. 4,833,734 issued to Der Estephanian on May 30, 1989 in class 2 and subclass 171 teaches a sweatband formed of an elastic fabric core of a length to resiliently encircle the head or wrist, and a low strength, highly absorbent paper fabric cover of excess length being gathered into a cushioning, absorbent pad by stitching onto the elastic fabric.

(4) U.S. Pat. No. 5,038,412 to Cionni.

U.S. Pat. No. 5,038,412 issued to Cionni on Aug. 13, 1991 in class 2 and subclass 209 teaches a headband with earmuffs where, in preferred form, the headband is fabricated from a stretchable material configured to incorporate a generally flat insulative pad interiorly of that band for each of the wearer's ears. Preferably, the headband is fabricated from a one-piece fabric blank folded upon itself to establish a generally tubular cross-sectional configuration, with the insulative pads stitched to that blank interiorly of the tubular headband.

(5) U.S. Pat. No. 5,745,921 to Mitchell et al.

U.S. Pat. No. 5,745,921 issued to Mitchell et al. on May 5, 1998 in class 2 and subclass 181 teaches a sweat band for absorbing and transferring moisture from the brow of a wearer's head, including an elongate body having a generally tubular medial portion for engaging the brow of a wearer's head and extending ends for securing the band about the wearer's head, with the medial portion thus positioned. The medial portion of the elongate body is formed of a porous cloth cover on the outer surface thereof, a layer of porous batting material disposed internally of the porous cloth cover in a generally tubular configuration, a surface skin on each side of the layer of porous batting material providing retention of and travel of moisture longitudinally of the layer of porous batting material, and securing elements retaining the layer of porous batting material and the surface skin in a predetermined position relative to the porous cloth cover.

(6) U.S. Pat. No. 5,930,842 to Burruss.

U.S. Pat. No. 5,930,842 issued to Burruss on Aug. 3, 1999 in class 2 and subclass 452 teaches an ear protection device made of an insulating fabric adjustably and releasably fastening to the retaining strap of an eye goggles unit. The device utilizes elastic apparatus to retain its lower edge proximate the head of the wearer under even severe conditions.

(7) U.S. Pat. No. 6,263,512 to LeDonne.

U.S. Pat. No. 6,263,512 issued to LeDonne on Jul. 24, 2001 in class 2 and subclass 171 teaches an article of apparel to be worn about the head, including an elongate sleeve of material having an elongate pliable member extending at least partially through the sleeve. The ends of the article are securable about the head so that at least a portion of the head and ears are covered, with the pliable member providing all or a portion of the facilities for securing and maintaining the device about the head.

(8) U.S. Pat. No. 6,502,245 to Mcbride.

U.S. Pat. No. 6,502,245 issued to Mcbride on Jan. 7, 2003 in class 2 and subclass 181 teaches a self-adjusting fabric covered sweatband designed to fit in any type of headwear to achieve a one size fits all situation. The sweatband is completely elastic all the way through its circumference and is covered by a fabric material to absorb the sweat.

It is apparent that numerous innovations for headbands have been provided in the prior art that are adapted to be used. Furthermore, even though these innovations may be suitable for the specific individual purposes to which they address, however, they would not be suitable for the purposes of the embodiments of the present invention as heretofore described, namely, a multi-function headband.

### 2. SUMMARY OF THE INVENTION

Thus, it is an object of the embodiments of the present invention to provide a multi-function headband, which avoids the disadvantages of the prior art.

Briefly stated, another object of the embodiments of the present invention is to provide a multi-function headband, including a head band, a pair of ear muffs, a neck protector, and apparatus for replaceably maintaining the head band around the head of a user. The head band encircles the head of the user. The pair of ear muffs depend from the head band and overlie and protect the ears of the user, respectively. The neck protector depends from the head band and overlies and protects the back-of-the-neck of the user.

The novel features which are considered characteristic of the embodiments of the present invention are set forth in the appended claims. The embodiments of the present invention themselves, however, both as to their construction and to their method of operation together with additional objects and advantages thereof will be best understood from the following description of the specific embodiments when read and understood in connection with the accompanying drawing.

### 3. BRIEF DESCRIPTION OF THE DRAWING

The figures of the drawing are briefly described as follows:

FIG. 1 is a diagrammatic perspective view of a first embodiment of the multi-function headband of the present invention in use;

FIG. 2 is an enlarged diagrammatic perspective view of the area generally enclosed by the dotted curve identified by ARROW 2 in FIG. 1;

FIG. 3 is an enlarged diagrammatic perspective view of the first embodiment of the multi-function headband of the present invention identified by ARROW 3 in FIG. 2;

FIG. 4 is a diagrammatic perspective view of a second embodiment of the multi-function headband of the present invention;

FIG. 5 is a diagrammatic perspective view of a third embodiment of the multi-function headband of the present invention;

FIG. 6 is a diagrammatic perspective view of a fourth embodiment of the multi-function headband of the present invention;

FIG. 6A is an enlarged diagrammatic cross sectional view taken along LINE 6A-6A in FIG. 6;

FIG. 6B is an enlarged diagrammatic cross sectional view taken along LINE 6B-6B in FIG. 6;

FIG. 6C is an enlarged diagrammatic cross sectional view taken along LINE 6C-6C in FIG. 6;

FIG. 7 is a diagrammatic perspective view of a fifth embodiment of the multi-function headband of the present invention in use;

FIG. 8 is an enlarged diagrammatic perspective view of the area generally enclosed by the dotted curve identified by ARROW 8 in FIG. 7;

FIG. 9 is an enlarged exploded diagrammatic perspective view of the fifth embodiment of the multi-function headband of the present invention identified by ARROW 9 in FIG. 8;

FIG. 9A is an enlarged diagrammatic cross sectional view taken along LINE 9A-9A in FIG. 9;

FIG. 10 is an exploded diagrammatic perspective view of a sixth embodiment of the multi-function headband of the present invention in use;

FIG. 11 is a diagrammatic perspective view of the sixth embodiment of the multi-function headband of the present invention in use;

FIG. 12 is a diagrammatic perspective view of a seventh embodiment of the multi-function headband of the present invention in use;

FIG. 13 is an exploded diagrammatic perspective view of the seventh embodiment of the multi-function headband of the present invention identified by ARROW 13 in FIG. 12; and

FIG. 14 is an enlarged diagrammatic cross sectional view taken along LINE 14-14 in FIG. 12.

### 4. LIST OF REFERENCE NUMERALS UTILIZED IN THE DRAWING

### A. General.

20 multi-function headband of present invention

### B. Configuration of First Embodiment of Multi-Function Headband 20.

22 head band for encircling head of user

24 pair of ear muffs for overlying and protecting ears of user, respectively

26 neck protector for overlying and protecting back-of-neck of user

28 apparatus for replaceably maintaining head band 22 around head of user

30 pair of free ends of head band 22 of apparatus 28

32 piece of release-sheet-protected tape of apparatus 28

34 inner surface of each free end of pair of free ends 30 of head band 22

36 outer surface of each free end of pair of free ends 30 of head band 22

### C. Configuration of Second Embodiment of Multi-Function Headband 40.

42 elastic band

44 uppermost terminus of neck-covering 26

### D. Configuration of Third Embodiment of Multi-Function Headband 50.

52 padding in ear muffs 24

54 hook and loop fasteners 54

### E. Configuration of Fourth Embodiment of Multi-Function Headband 60.

62 pair of elastic bands

64 sweat-absorbent pad for overlying brow of user

65 uppermost terminus of ear muffs 24

66 inner surface of head band 22

68 end of each band of pair of elastic bands 68

### F. Configuration of Fifth Embodiment of Multi-Function Headband 70.

72 nose protector

74 head band portion of nose protector 72 for overlying brow of user

76 nose portion of nose protector 72 for overlying and protecting nose of user

78 adhesive

### G. Configuration of Sixth Embodiment of Multi-Function Headband 80.

82 eye protector for overlying and protecting eyes of user

84 head protector for overlying and protecting head of user

86 hook and loop fasteners

88 hook and loop fasteners

90 hook and loop fasteners

92 front of head band 22

94 back of head band 22

### H. Configuration of Seventh Embodiment of Multi-Function Headband 100.

102 secondary strap for encircling neck of user

104 posterior pocket for overlying back-of-neck of user.

106 lateral pockets for overlying sides-of-neck of user

108 gel packs

110 pair of free ends of secondary strap 102

112 piece of release-sheet-protected tape

114 inner surface of each free end of pair of free ends 110 of secondary strap 102

116 outer surface of each free end of pair of free ends 110 of secondary strap 102

118 inner surface of neck protector 26

120 inner surfaces of secondary strap 102

### 5. DETAILED DESCRIPTION OF THE PREFERRED EMBODIMENTS

### A. General.

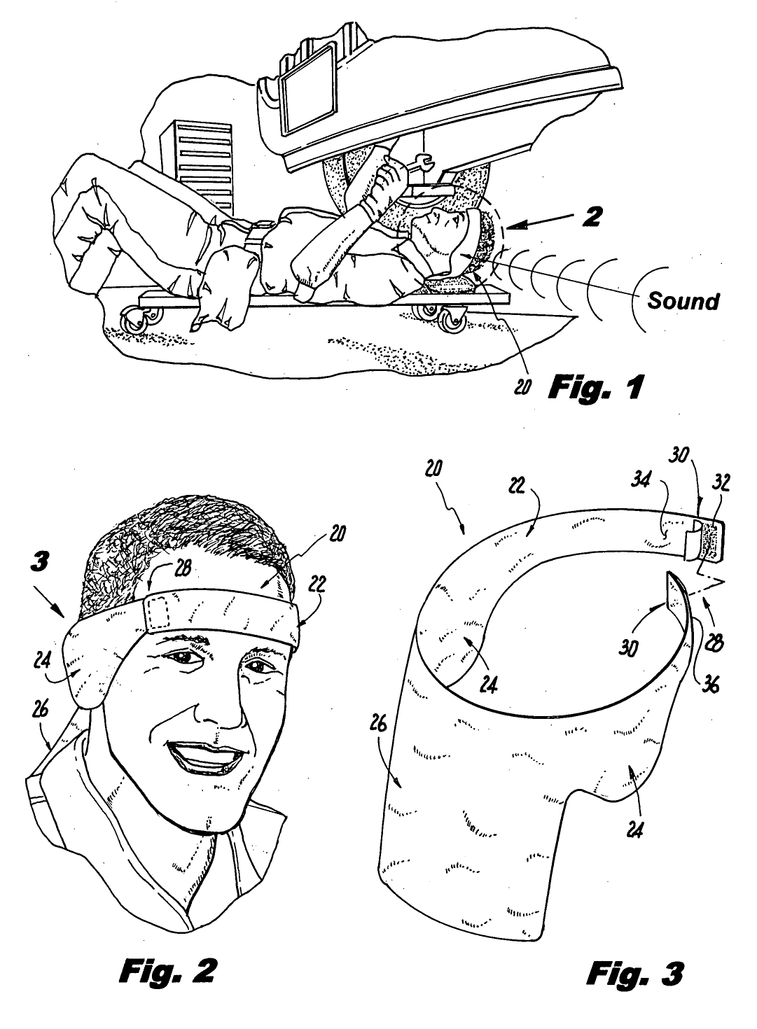

Referring now to the figures, in which like numerals indicate like parts, and particularly to FIGS. 1 and 2, which are, respectively, a diagrammatic perspective view of a first embodiment of the multi-function headband of the present invention in use, and an enlarged diagrammatic perspective view of the area generally enclosed by the dotted curve identified by ARROW 2 in FIG. 1, the first embodiment of the multi-function headband of the present invention is shown generally at 20.

### B. The Configuration of the First Embodiment of the Multi-Function Headband 20.

The multi-function headband 20 comprises a head band 22, a pair of ear muffs 24, a neck protector 26, and apparatus 28 for replaceably maintaining the head band 22 around the head of a user.

The head band 22 is for encircling the head of the user. The pair of ear muffs 24 depend from the head band 22 and are for overlying and protecting the ears of the user, respectively. The neck protector 26 depends from the head band 22 and is for overlying and protecting the back-of-the-neck of the user.

The head band 22, the pair of ear muffs 24, and the neck protector 26 are one-piece.

The apparatus 28 includes the head band 22 terminating in a pair of free ends 30, and a piece of release-sheet-protected tape 32. Each free end 30 of the head band 22 has an inner surface 34 and an outer surface 36. The piece of release-sheet-protected tape 32 of the apparatus 28 is disposed on the inner surface 34 of one free end 30 of the head band 22 and selectively engages the outer surface 36 of the other free end 30 of the head band 22.

### C. The Configuration of a Second Embodiment of the Multi-Function Headband 40.

The configuration of the second embodiment of the multi-function headband 40 can best be seen in FIG. 4, which is a diagrammatic perspective view of a second embodiment of the multi-function headband of the present invention, and as such, will be discussed with reference thereto.

The multi-function headband 40 is similar to the multi-function head band 20, but with the addition of an elastic band 42. The elastic band 42 is disposed in the head band 22, at an uppermost terminus 44 of the neck-covering 26.

### D. The Configuration of a Third Embodiment of the Multi-Function Headband 50.

The configuration of the third embodiment of the multi-function headband 50 can best be seen in FIG. 5, which is a diagrammatic perspective view of a third embodiment of the multi-function headband of the present invention, and as such, will be discussed with reference thereto.

The multi-function headband 50 is similar to the multi-function headband 20, but with the neck-covering 26 being separate from, and attached to, the head band 22, instead of one-piece therewith, and the addition of padding 52 in the ear muffs 24 and interlocking free ends 30, and the piece of release-sheet-protected tape 32 of the apparatus 28 being replaced by hook and loop fasteners 54.

### E. The Configuration of a Fourth Embodiment of the Multi-Function Headband 60.

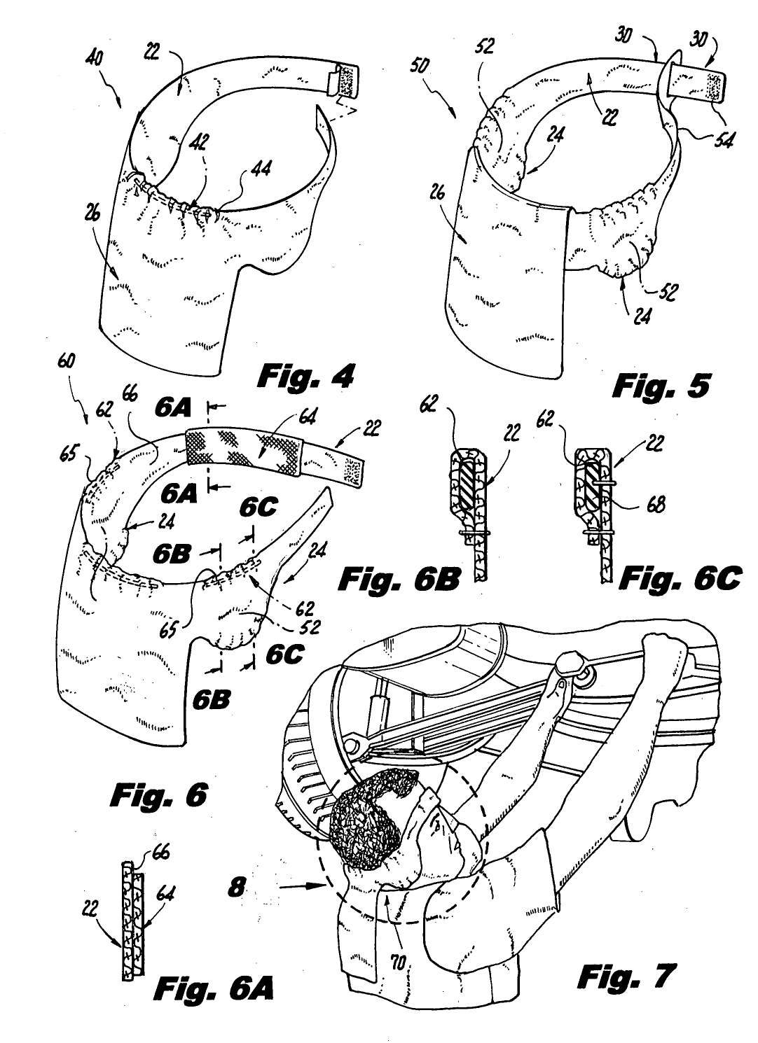

The configuration of a fourth embodiment of the multi-function headband 60 can best be seen in FIGS. 6 and 6A-6C, which are, respectively, a diagrammatic perspective view of a fourth embodiment of the multi-function headband of the present invention, an enlarged diagrammatic cross sectional view taken along LINE 6A-6A in FIG. 6, an enlarged diagrammatic cross sectional view taken along LINE 6B-6B in FIG. 6, and an enlarged diagrammatic cross sectional view taken along LINE 6C-6C in FIG. 6, and as such, will be discussed with reference thereto.

The multi-function headband 60 is similar to the multi-function headband 40, but with the addition of the padding 52 in the ear muffs 24, a pair of elastic bands 62, and a sweat-absorbent pad 64. The pair of elastic bands 62 are disposed in the head band 22, at an uppermost terminus 65 of the ear muffs 24.

As shown in FIG. 6A, the sweat-absorbent pad 64 is fixedly attached to an inner surface 66 of the head band 22 and is for overlying the brow of the user.

As shown in FIG. 6B, each elastic band 62 extends freely in the head band 22, over an associated ear muff 24, and as shown in FIG. 6C, has an end 68 thereof affixed in the head band 22.

### F. The Configuration of a Fifth Embodiment of the Multi-Function Headband 70.

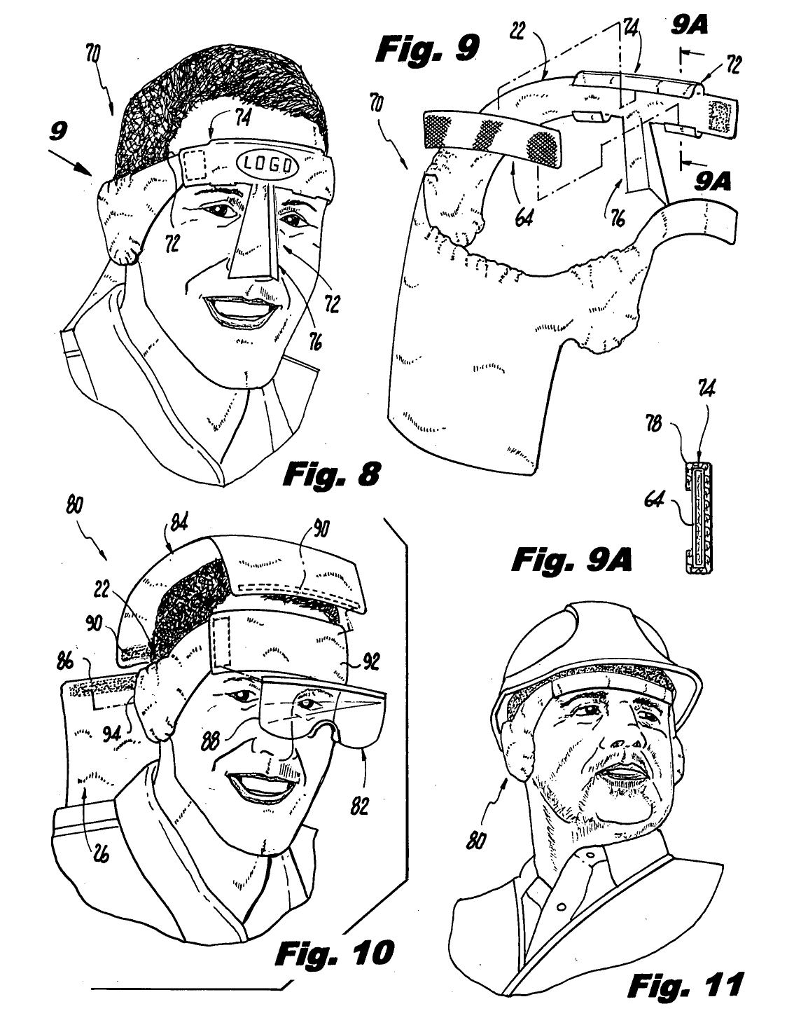

The configuration of the multi-function headband 70 can best be seen in FIGS. 7-9 and 9A, which are, respectively, a diagrammatic perspective view of a fifth embodiment of the multi-function headband of the present invention in use, an enlarged diagrammatic perspective view of the area generally enclosed by the dotted curve identified by ARROW 8 in FIG. 7, an enlarged exploded diagrammatic perspective view of the fifth embodiment of the multi-function headband of the present invention identified by ARROW 9 in FIG. 8, and an enlarged diagrammatic cross sectional view taken along LINE 9A-9A in FIG. 9, and as such, will be discussed with reference thereto.

The multi-function headband 70 is similar to the multi-function head band 60, but with the addition of a nose protector 72. The nose protector 72 has a head band portion 74 and a nose portion 76. The head band portion 74 of the nose protector 72 overlies the head band 22 for overlying the brow of the user, and wraps around the head band 22 for facing the brow of the user, and is maintained thereat, by the sweat-absorbent pad 64 being affixed thereto by an adhesive 78 (FIG. 9A). The nose portion 76 of the nose protector 72 depends from the head band portion 74 of the nose protector 72 and is hard for overlying and protecting the nose of the user.

### G. The Configuration of a Sixth Embodiment of the Multi-Function Headband 80.

The configuration of the sixth embodiment of the multi-function headband 80 can best be seen in FIGS. 10 and 11, which are, respectively, an exploded diagrammatic perspective view of a sixth embodiment of the multi-function headband of the present invention in use, and a diagrammatic perspective view of the sixth embodiment of the multi-function headband of the present invention in use, and as such, will be discussed with reference thereto.

The multi-function head band 80 is similar to that of any of the multi-head band 20, 40, 50, 60, or 70, but with the addition of an eye protector 82 and a head protector 84, and the neck protector 26 being replaceably attached to the head band 22 by hook and loop fasteners 86.

The eye protector 82 is replaceably affixed by hook and loop fasteners 88 to the head band 22 and is for overlying and protecting the eyes of the user.

The head protector 84 is flexible and replaceably affixed by hook and loop fasteners 90 to the head band 22, at a front 92 and a back 94 thereof, and is for overlying and protecting the head of the user.

### H. The Configuration of a Seventh Embodiment of the Multi-Function Headband 100.

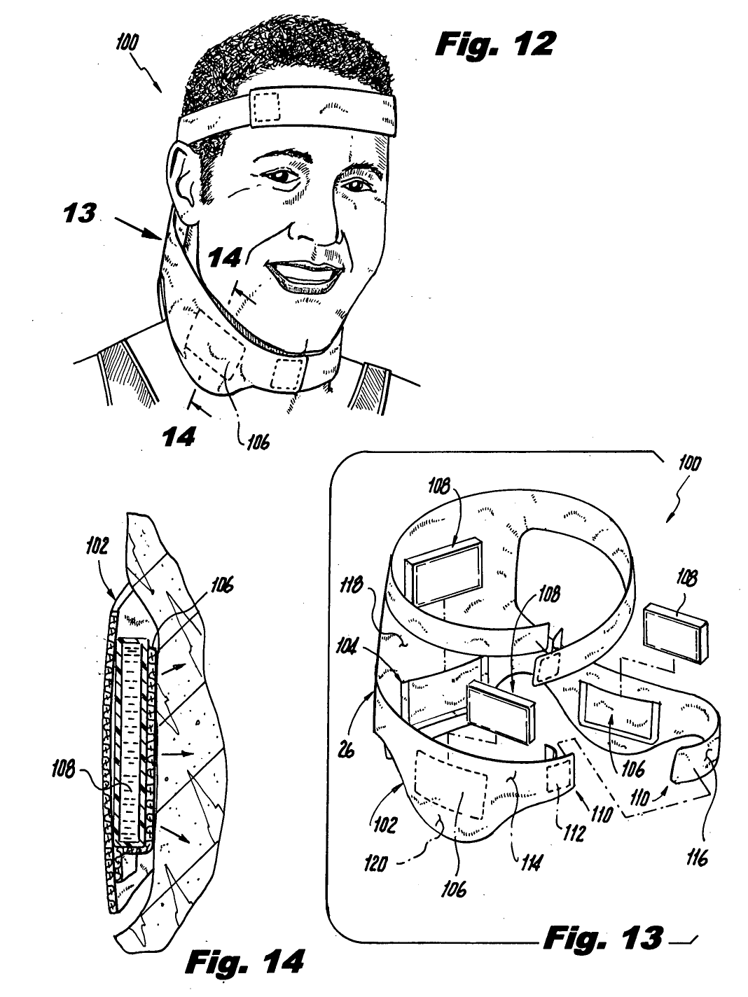

The configuration of the multi-function head band 100 can best be seen in FIGS. 12-14, which are, respectively, a diagrammatic perspective view of a seventh embodiment of the multi-function headband of the present invention in use, an exploded diagrammatic perspective view of the seventh embodiment of the multi-function headband of the present invention identified by ARROW 13 in FIG. 12, and an enlarged diagrammatic cross sectional view taken along LINE 14-14 in FIG. 12, and as such, will be discussed with reference thereto.

The multi-function head band 100 is similar to the multi-function head band 20, but with the addition of a secondary strap 102, a posterior pocket 104, lateral pockets 106, and gel packs 108.

The secondary strap 102 extends from the neck protector 26, is for encircling the neck of the user, and terminates in a pair of free ends 110 and has a piece of release-sheet-protected tape 112. Each free end 110 of the secondary strap 102 has an inner surface 114 and an outer surface 116. The piece of release-sheet-protected tape 112 is disposed on the inner surface 114 of one free end 110 of the secondary strap 102 and selectively engages the outer surface 116 of the other free end 110 of the secondary strap 102.

The posterior pocket 104 is disposed on an inner surface 118 of the neck protector 26 and is for overlying the back-of-the-neck of the user.

The lateral pockets 106 are disposed on inner surfaces 120 of the secondary strap 102 and are for overlying the sides-of-the-neck of the user.

The gel backs 108 can be either hot or cold and are replaceably inserted into any of the posterior pocket 104 and the lateral pockets 106.

### I. The Conclusions.

It will be understood that each of the elements described above or two or more together may also find a useful application in other types of constructions differing from the types described above.

While the embodiments of the present invention have been illustrated and described as embodied in a multi-function headband, however, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt them for various applications without omitting features that from the standpoint of prior art fairly constitute characteristics of the generic or specific aspects of the embodiments of the present invention.

The invention claimed is:

1. A multi-function headband, comprising;
a) a head band;
b) a pair of ear muffs;
c) a neck protector; and
d) means for replaceably maintaining said head band around the head of a user;
wherein said head band is for encircling the head of the user;
wherein said pair of ear muffs depend from said head band;
wherein said pair of ear muffs are for overlying and protecting the ears of the user, respectively;
wherein said neck protector depends from said head band; and
wherein said neck protector is for overlying and protecting the back-of-the-neck of the user.

2. The headband of claim 1, wherein said head band, said pair of ear muffs, and said neck protector are one-piece.

3. The headband of claim 1, wherein said means includes said head band terminating in a pair of free ends;
wherein said means includes a piece of release-sheet-protected tape;
wherein each free end of said head band has an inner surface;
wherein each free end of said head band has an outer surface; and
wherein said piece of release-sheet-protected tape is disposed on said inner surface of one free end of said head band and selectively engages said outer surface of the other free end of said head band.

4. The headband of claim 3, further comprising an elastic band;
wherein said elastic band is disposed in said head band; and
wherein said elastic band is disposed at an uppermost terminus of said neck-covering.

5. The headband of claim 3, wherein said neck-covering is separate from said head band; and
wherein said neck-covering is attached to said head band.

6. The headband of claim 5, wherein said ear muffs contain padding.

7. The headband of claim 6, wherein said means includes said head band having interlocking free ends; and
wherein said means includes hook and loop fasteners.

8. The headband of claim 4, further comprising a pair of elastic bands;
wherein said pair of elastic bands are disposed in said head band; and
wherein said pair of elastic bands are disposed at an uppermost terminus of said ear muffs, respectively.

9. The headband of claim 8, further comprising a sweat-absorbent pad;
wherein said sweat-absorbent pad is fixedly attached to an inner surface of said head band; and
wherein said sweat-absorbent pad is for overlying the brow of the user.

10. The headband of claim 9, wherein said ear muffs contain padding.

11. The headband of claim 8, wherein each elastic band extends freely in said head band;
wherein each elastic band extends over an associated ear muff; and
wherein each elastic band has an end thereof affixed in said head band.

12. The headband of claim 11, further comprising a nose protector.

13. The headband of claim 12, wherein said nose protector has a head band portion;
wherein said nose protector has a nose portion;
wherein said head band portion of said nose protector overlies said head band;
wherein said head band portion of said nose protector is for overlying the brow of the user;
wherein said head band portion of said nose protector wraps around said head band for facing the brow of the user, and is maintained thereat, by said sweat-absorbent pad being affixed thereto by an adhesive;
wherein said nose portion of said nose protector depends from said head band portion of said nose protector; and
wherein said nose portion of said nose protector is hard for overlying and protecting the nose of the user.

14. The headband of claim 3, 4, 7, 11, 12, or 13, further comprising an eye protector;
wherein said eye protector is replaceably affixed by hook and loop fasteners to said head band; and
wherein said eye protector is for overlying and protecting the eyes of the user.

15. The headband of claim 14, further comprising a head protector;
wherein said head protector is replaceably affixed by hook and loop fasteners to said head band, at a front and a back thereof; and
wherein said head protector is for overlying and protecting the head of the user.

16. The headband of claim 15, wherein said neck protector is replaceably attached to said head band by hook and loop fasteners.

17. The headband of claim 1, further comprising a secondary strap;
wherein said secondary strap extends from said neck protector;
wherein said secondary strap is for encircling the neck of the user;
wherein said secondary strap terminates in a pair of free ends;
wherein each free end of said secondary strap has an inner surface; and
wherein each free end of said secondary strap has an outer surface.

18. The headband of claim 17, further comprising a posterior pocket;
wherein said posterior pocket is disposed on an inner surface of said neck protector; and
wherein said posterior pocket is for overlying the back-of-the-neck of the user.

19. The headband of claim 18, further comprising lateral pockets;
wherein said lateral pockets are disposed on inner surfaces of said secondary strap; and
wherein said lateral pockets are for overlying the sides-of-the-neck of the user, respectively.

20. The headband of claim 19, further comprising gel packs; and
wherein said gel packs are replaceably inserted into at least one of said posterior pocket and said lateral pockets.

21. The headband of claim 20, wherein said means includes a piece of release-sheet-protected tape;
wherein said piece of release-sheet-protected tape is disposed on said inner surface of one free end of said secondary strap; and
wherein said piece of release-sheet-protected tape selectively engages said outer surface of the other free end of said secondary strap.
